(19) United States
(12) Patent Application Publication
(10) Pub. No.: US 2023/0278979 A1
(43) Pub. Date: Sep. 7, 2023

(54) SALTS OF A DIHYDROOROTATE DEHYDROGENASE (DHOD) INHIBITOR

(71) Applicant: KIORA PHARMACEUTICALS GmbH, Vienna (AT)

(72) Inventors: Stefan SPERL, Wien (AT); Lisa PLASSER, Wien (AT)

(21) Appl. No.: 18/172,722

(22) Filed: Feb. 22, 2023

Related U.S. Application Data
(60) Provisional application No. 63/315,316, filed on Mar. 1, 2022.

(51) Int. Cl. C07D 333/40

(52) U.S. Cl. CPC C07D 333/40

(57) ABSTRACT

The present disclosure provides salts of 3-(2,3,5,6-tetrafluoro-3′-trifluoromethoxy-biphenyl-4-ylcarbamoyl)-thiophene-2-carboxylic acid. The present disclosure also provides pharmaceutical compositions comprising salts of the invention, and methods of treating, preventing, or ameliorating a disease or condition comprising administering a salt of the invention.

### FIELD OF THE INVENTION

The present disclosure provides salts of 3-(2,3,5,6-tetrafluoro-3′-trifluoromethoxy-biphenyl-4-ylcarbamoyl)-thiophene-2-carboxylic acid (PP-001). The present disclosure also provides pharmaceutical compositions comprising salts of PP-001, and methods of treating, preventing, or ameliorating a disease or condition comprising administering a salt of PP-001.

### BACKGROUND

The small molecule compound 3-(2,3,5,6-tetrafluoro-3′-trifluoromethoxy-biphenyl-4-ylcarbamoyl)-thiophene-2-carboxylic acid (referred to herein as PP-001), shown below is a potent dihydroorotate dehydrogenase (DHODH) inhibitor.

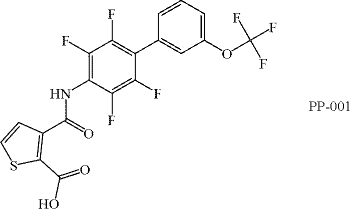 [SMILES: O=C(Nc1c(F)c(F)c(-c2cccc(OC(F)(F)F)c2)c(F)c1F)c1ccsc1C(=O)O]

See, e.g., US 7,365,094, US 8,354,433 and US 9,795,590, incorporated herein by reference. PP-001 has found application in treating diseases and conditions associated with DHOD activity.

### SUMMARY OF THE INVENTION

In some embodiments, the disclosure provides a compound of Formula I

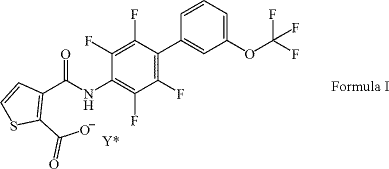

wherein Y* is
i) a single atom cation with a +1 charge;
ii) a single atom cation with a +2 charge;
iii) a carboxyalkylammonium cation, optionally substituted with one or more hydroxyl or amino groups;
iv) a dialkylammonium cation, optionally substituted with one or more hydroxyl groups; or
v) an alkylammonium cation, optionally substituted with one or more hydroxyl groups.

In some embodiments, the Y* is a single atom cation with a +1 charge. In some embodiments, the single atom cation with a +1 charge is a sodium cation or a potassium cation.

In some embodiments, Y* is a single atom cation with a +2 charge. In some embodiments, the single atom cation with a +2 charge is a calcium cation, a magnesium cation, or a zinc cation.

In some embodiments, the Y* is a carboxyalkylammonium cation. In some embodiments, the carboxyalkylammonium cation is a carboxy(C₃-C₆) alkylammonium cation. In some embodiments, the carboxy(C₃-C₆)alkylammonium cation is a carboxypentylammonium cation. In some embodiments, the carboxypentylammonium cation is substituted with one amino group. In some embodiments, the carboxypentylammonium cation is lysine.

In some embodiments, the Y* is a dialkylammonium cation. In some embodiments, the dialkylammonium cation is a (C₃-C₆)dialkylammonium cation. In some embodiments, the (C₃-C₆)dialkylammonium cation is a hexylammoniumalkyl cation. In some embodiments, the hexylammoniumalkyl cation is a hexylammoniummethyl cation. In some embodiments, the hexylammoniummethyl cation is substituted with four to five hydroxyl groups. In some embodiments, the hexylammoniummethyl cation is meglumine.

In some embodiments, the Y* is an alkylammonium cation. In some embodiments, the alkylammonium cation is a (C₃-C₆)alkylammonium cation. In some embodiments, the (C₃-C₆)alkylammonium cation is a butylammonium cation. In some embodiments, the butylammonium cation is a tert-butylammonium cation. In some embodiments, the tert-butylammonium cation is substituted with two to three hydroxyl groups. In some embodiments, the tert-butylammonium cation is tromethamine.

In some embodiments, the solubility of the compound of Formula I is greater than 1 mg/ml at 37° C., pH 6.4 to 6.5 and 1 atm pressure.

In some embodiments, the bioavailability of the compound of Formula I in a dog is greater than 20%. In some embodiments, the bioavailability of the compound of Formula I in a dog is between 20% to 99%. In some embodiments, the bioavailability of the compound of Formula I in a dog is between 40% to 95%.

In some embodiments, the bioavailability of the compound of Formula I in a dog is at least 2-fold greater than the free acid form of the compound.

In some embodiments, the disclosure provides a pharmaceutical composition comprising: a compound of Formula I as described herein and a pharmaceutically acceptable excipient, carrier, diluent, or combination thereof.

In some embodiments, the disclosure provides a method of treating an inflammatory disease or an autoimmune disease comprising administering to a subject in need thereof an effective amount of pharmaceutically acceptable composition comprising a compound of Formula I as described herein and a pharmaceutically acceptable excipient, carrier, diluent, or combination thereof. In some embodiments, the administering is by oral administration.

In some embodiments, the disclosure provides a compound of Formula I as described herein, wherein Y* is a potassium cation.

In some embodiments, the disclosure provides a compound of Formula I as described herein, wherein the bioavailability of the compound of Formula I in a dog is greater than 20%.

In some embodiments, the disclosure provides a compound of Formula I as described herein, wherein the solubility of the compound of Formula I is greater than 1 mg/ml at pH 6.4 to 6.5, at 37° C., in a FaSSIF media.

### BRIEF DESCRIPTION OF THE DRAWINGS

FIG. 1 shows the solubility over time of various PP-001 salts.

### DETAILED DESCRIPTION OF THE INVENTION

The present disclosure relates to salts of 3-(2,3,5,6-tetrafluoro-3′-trifluoromethoxy-biphenyl-4-ylcarbamoyl)-thiophene-2-carboxylic acid.

Different salt forms of a given compound may have different properties, such as solubility, dissolution rate, suspension stability, stability during milling, vapor pressure, optical and mechanical properties, hygroscopicity, crystal size, filtration performance, drying, density, melting point, degradation stability, stability to prevent phase change to other forms, color and even chemical reactivity. More importantly, the different salt forms of a small molecule compound may change its dissolution, dissolution performance, pharmacokinetics and bioavailability, which will affect the efficacy and safety performance of a drug.

In particular, in some embodiments salt forms of a drug can affect its dissolution, absorption in vivo, thereby affecting its clinical therapeutic effect and safety to a certain extent. In some embodiments, for some slightly soluble solid or semisolid oral preparations, the influence of salt forms can be critical. The present disclosure has identified various salts of PP-001 which have beneficial properties for various uses and indications.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.,” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As used herein, the term “room temperature” generally refers to about 15° C. to about 32° C. In some embodiments, the term refers to about 18° C. to about 22° C. In some embodiments, the term refers to 20±5° C.

As used herein, the term “pharmaceutically acceptable” excipient, carrier, diluent, or ingredient refers to a substance that is suitable for use in humans and/or animals without excessive adverse side effects (such as toxicity, irritation, and allergies), that is, with a reasonable benefit/risk ratio.

As used herein, the term “pharmaceutically acceptable ingredient” refers to a substance that is suitable for use in humans and/or animals without excessive adverse side effects (such as toxicity, irritation, and allergies), that is, with a reasonable benefit/risk ratio.

As used herein, the term “effective amount” refers to an amount of a therapeutic agent to treat, alleviate or prevent a target disease or condition, or an amount that exhibits a detectable therapeutic or preventive effect. The exact effective amount for a subject depends on the subject’s size and health, the nature and extent of the condition, and the chosen therapeutic agent and/or combination of therapeutic agents. Therefore, it is not useful to specify an accurate effective amount in advance. However, for a given condition, a routine experiment can be used to determine the effective amount, which can be judged by the clinician.

As used herein, the term “administration” or “administering” refers to routes of introducing a compound or composition provided herein to an individual to perform its intended function. An example of a route of administration that can be used includes, but is not limited to, parenteral administration, such as subcutaneous, intravenous, or intramuscular injection or infusion, or oral administration.

In some embodiments, the disclosure provides a compound of Formula I

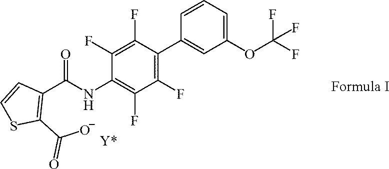

wherein Y* is
i) a single atom cation with a +1 charge;
ii) a single atom cation with a +2 charge;
iii) a carboxyalkylammonium cation, optionally substituted with one or more hydroxyl or amino groups;
iv) a dialkylammonium cation, optionally substituted with one or more hydroxyl groups; or
v) an alkylammonium cation, optionally substituted with one or more hydroxyl groups.

As used herein, the term compound refers to the small molecule (e.g., PP-001) in combination with the indicated cation (Y*). For example, in some embodiments, the compound is of Formula I.

Various cations, i.e., Y*, of PP-001 can be used. The disclosure herein provides the beneficial properties of various cations. The term “salts” and the “cations” described herein are interchangeable. Thus, the phrase “salt of PP-001”, would include the PP-001 and the cations described herein. In some embodiments, the Y* is a charged single atom. For example, in some embodiments, the Y* is a positively charged cation. In some embodiments, the single atom cation has a +1 charge. One of ordinary skill in the art will appreciate that the “charge” of an atom or compound can be dependent on a number of factors, for example the pH and the temperature. One of ordinary skill in the art will appreciate that the charge will be affected by the pH according to the pKa values of the atom or compound. As used herein, unless otherwise specified, when referring to the charge of Y*, the pH is about 6 to 8. In some embodiments, the pH is about 6.4 to 7.5. In some embodiments, the pH is about 6.4 to 6.5. In some embodiments, the temperature is 37° C.

In some embodiments, the single atom cation with a +1 charge is a lithium cation, sodium cation, a potassium cation, and rubidium cation. In some embodiments, the single atom cation with a +1 charge is a sodium cation and a potassium cation. In some embodiments, the single atom cation with a +1 charge is a sodium cation. In some embodiments, the single atom cation with a +1 charge is a potassium cation.

In some embodiments, the disclosure provides a compound of Formula I as described herein, wherein Y* is a potassium ion.

In some embodiments, Y* is a single atom cation with a +2 charge. In some embodiments, the single atom cation with a +2 charge is a copper cation, an iron cation, a calcium cation, a magnesium cation, or a zinc cation. In some embodiments, the single atom cation with a +2 charge is a calcium cation, a magnesium cation, or a zinc cation.

In some embodiments, the Y* is a carboxyalkylammonium cation, optionally substituted with one or more hydroxyl or amino groups. The term carboxyalkylammonium refers to a compound with the generic formula of

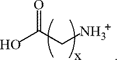

In some embodiments, x is 1 to 15. In some embodiments, x is 1 to 8. In some embodiments, x is 1 to 6. In some embodiments, x is 1, 2, 3, 4, 5 or 6.

Unless specified otherwise, the term alkyl as used herein, when used alone or in combination with other groups or atoms, refers to a saturated straight or branched chain consisting of 1 to 15 hydrogen-substituted carbon atoms. The skilled artisan reading the present disclosure in its entirety will recognize that “x” can refer to both linear and branched carbons, even though the generic formula only denotes linear carbons throughout. In some embodiments, the term alkyl comprises 1 to 8 carbons. In some embodiments, the term alkyl comprises 3 to 6 carbons. In some embodiments, the term alkyl comprises 1, 2, 3, 4, 5, or 6 carbons. In some embodiments, the alkyl can be linear. In some embodiments, the alkyl can be branched. In some embodiments, the term refers to methyl, ethyl, propyl, isopropyl, n-butyl, 1-methylpropyl, isobutyl, t-butyl, 2,2-dimehylbutyl, n-pentyl, 2-methylpentyl, 3-methylpentyl, 4-methylpentyl, n-hexyl. In some embodiments, the alkyl group can be substituted with a methyl, ethyl, hydroxyl groups, amino groups or halogen, e.g., chloro or fluoro.

In some embodiments, the carboxyalkylammonium cation is a carboxy(C₃-C₆)alkylammonium cation. In some embodiments, the carboxyalkylammonium cation is a carboxypentylammonium cation, a carboxybutylammonium cation, or a carboxypropylammonium cation, optionally substituted with a methyl, ethyl, hydroxyl groups, amino groups or halogen, e.g., chloro or fluoro. In some embodiments, the carboxyalkylammonoim cation is a carboxypentylammonium cation, a carboxybutylammonium cation, or a carboxypropylammonium cation, optionally substituted with an ammonium group. In some embodiments, the carboxy(C₃-C₆)alkylammonium cation is a carboxypentylammonium cation. In some embodiments, the carboxypentylammonium cation is substituted with one or more hydroxyl group. In some embodiments, the carboxypentylammonium cation is substituted with one or more ammonium groups. In some embodiments, the carboxypentylammonium cation is substituted with one ammonium group. In some embodiments, the carboxypentylammonium cation is lysine.

In some embodiments, the Y* is a dialkylammonium cation, optionally substituted with one or more hydroxyl groups. The term dialkylammonium refers to a compound with the generic formula of

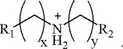

In some embodiments, x is 1 to 15. In some embodiments, x is 1 to 8. In some embodiments, x is 1 to 6. In some embodiments, x is 1, 2, 3, 4, 5 or 6. In some embodiments, x is 3 to 6. In some embodiments, y is 1 to 15. In some embodiments, y is 1 to 8. In some embodiments, y is 3 to 6. In some embodiments, y is 1 to 6. In some embodiments, y is 1, 2, 3, 4, 5 or 6. In some embodiments, x is 1 to 6 and y is 1. In some embodiments, x is 5 or 6 and y is 1. In some embodiments, R₁ is a hydrogen atom or a hydroxyl group. In some embodiments, R₂ is a hydrogen atom or a hydroxyl group.

In some embodiments, the dialkylammonium cation is a (C₁-C₆)dialkylammonium cation. In some embodiments, the alkyl chains in the dialkylammonium cation can be the same or be different, e.g., a propylammoniummethyl cation or a butylammoniumethyl cation. In some embodiments, the (C₁-C₆)dialkylammonium cation is a hexylammoniumalkyl cation. In some embodiments, the hexylammoniumalkyl cation is a hexylammoniummethyl cation. In some embodiments, the hexylammoniummethyl cation is substituted with a methyl, ethyl, hydroxyl groups, amino groups or halogen, e.g., chloro or fluoro. In some embodiments, the hexylammoniummethyl cation is substituted with a hydroxyl group. In some embodiments, the hexylammoniummethyl cation is substituted with four to five hydroxyl groups. In some embodiments, the hexylammoniummethyl cation is substituted with five hydroxyl groups. In some embodiments, the hexylammoniummethyl cation is meglumine.

In some embodiments, the Y* is an alkylammonium cation, optionally substituted with one or more hydroxyl groups. The term alkylammonium cation refers to a compound with the generic formula of

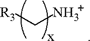

In some embodiments, x is 1 to 15. In some embodiments, x is 1 to 8. In some embodiments, x is 3 to 6. In some embodiments, R₃ is a hydrogen atom or a hydroxyl group. In some embodiments, the term alkylammonium refers to a compound branched alkylammoinium.

In some embodiments, the alkylammonium cation is a (C₃-C₆)alkylammonium cation. For example, in some embodiments, the alkylammonium cation is a propylammonium, isopropylammonium, butylammonium, 2′-butylammonium, tert-butylammonium, pentylammonium, 2′-pentylammonium, isopentylammonium cation, etc. In some embodiments, the alkylammonium cation is substituted with a methyl, ethyl, hydroxyl groups, ammonium groups or halogen, e.g., chloro or fluoro. In some embodiments, the alkylammonium cation is substituted with a hydroxyl group. In some embodiments, the (C₃-C₆)alkylammonium cation is a butylammonium cation. In some embodiments, the butylammonium cation is a tert-butylammonium cation. In some embodiments, the tert-butylammonium cation is substituted with a methyl, ethyl, hydroxyl group, amino group or halogen, e.g., chloro or fluoro. In some embodiments, the tert-butylammonium cation is substituted with two to three hydroxyl groups. In some embodiments, the tert-butylammonium cation is substituted with three hydroxyl groups. In some embodiments, the tert-butylammonium cation is tromethamine.

In some embodiments, the disclosure provides a compound of Formula I wherein Y* is a single atom cation with a +1 charge. In some embodiments, the disclosure provides a compound of wherein Y* is a potassium cation.

In some embodiments, the compounds of formula I have increased solubility relative to a non-salt form of PP-001. As used herein, the terms “non-salt form of PP-001” and “free-acid form” are interchangeable and refer to the PP-001 compound without an accompanying salt, and can include both PP-001 or the deprotonated charged form of PP-001 without the presence of a counter cation. One of skill in the art will appreciate that whether PP-001 is protonated or deprotonated will depend on a number of factors, including the pH and temperature of its environment.

In some embodiments, the increased solubility of the salt forms described herein allow the PP-001 compound to be readily solubilized. One of skill in the art will appreciate that solubility in a given solvent is dependent on a number of factors, including but not limited to the identity of the solvent, the temperature, the pH, the pressure, etc. Solubility as defined herein is the capacity of the solvent to dissolve the solute, i.e., PP-001. Solubility may be stated in units of concentration such as molality, molarity, mole fraction, mole ratio, weight/volume, or weight/weight. As used herein, unless designated otherwise, solubility is designated in a weight/volume concentration.

In some embodiments, solubility can be expressed in absolute as well as relative terms. As used herein, in relative terms, solubility of the given PP-001 salt can be compared to the solubility of PP-001 in non-salt form. By way of example only to exemplify how to calculate “increased solubility,” if the solubility of a given PP-001 salt is “1.2 mg/ml” and the solubility of PP-001 in non-salt form is “1.0 mg/mL,” then the increased solubility would 20%, i.e., (1.2 mg/mL - 1.0 mg/mL)/1.0 mg/mL. In some embodiments, the solubility of the compounds as described herein is greater than 5%, greater than 10%, greater than 20%, greater than 30%, greater than 40% or greater than 50% relative to the non-salt form.

Equilibrium solubility is the concentration limit, at thermodynamic equilibrium, to which a solute may be uniformly dissolved into a solvent when excess solid is present. The apparent solubility may be either higher or lower than the equilibrium solubility due to transient supersaturation or incomplete dissolution due to insufficient time to reach equilibrium. Equilibrium can be defined as sufficiently converged when it no longer changes significantly during a certain time frame. In some embodiments, the compounds of Formula I have an increased equilibrium solubility relative to equilibrium solubility of PP-001 in non-salt form.

In some embodiments, the dissolution rate whereas dissolution rate is how quickly the solubility limit is reached. In some embodiments, the compounds described herein have an increased dissolution rate in a given solvent relative to the dissolution rate of PP-001 in non-salt form.

In some embodiments, PP-001 may be more advantageous if the compound has reduced solubility. For example, in some embodiments, decreased solubility may be desired if extended release of PP-001 is desired. In such circumstances, a given salt of PP-001 may be selected to decrease solubility in a given environment.

In some embodiments, the compounds of Formula I have increased solubility when placed in various solvents, e.g., water, a buffer (e.g., a phosphate buffer), a media (e.g., a Fasted State Simulated Intestinal Fluid (FaSSIF) media or simulated gastric fluid (SGF) media), organic solvent, etc. In some embodiments, the solvent further comprises a co-solvent. In some embodiments, the co-solvent is water. Likewise, in some embodiments, when determining solubility, the solvent can comprise other salts or counter-ions which may further alter the solubility of the compounds of Formula I.

The solubility of ionizable acids and bases is pH dependent. In some embodiments, this pH dependence is due to the charged species have a higher affinity for the aqueous environment than the neutral form. In some embodiments, the Henderson-Hasselbach equation can be used to determine the increase in the solubility of the solute for changes in pH of the solution relative to the pKa (acidic) or pKa (basic) of the ionizable solute (acid or base). In some embodiments, when determining solubility, the pH of the solvent and/or the solution comprising the solvent and the solute (e.g., the compounds described herein) is about 5.0 to about 8.0, about 5.5 to about 7.5, about 6.0 to about 7.0 or about 6.4 to 6.5.

In some embodiments, the temperature at which solubility is determined can affect the solubility calculation. In some embodiments, solubility is determined at about 18° C. to about 45° C., or about 20° C. to about 28° C. In some embodiments, solubility is determined at about 24° C. to about 26° C. or about 25° C. In some embodiments, solubility is determined at about 30° C. to about 40° C. or about 37° C. In some embodiments solubility is determined under negative pressure, 1 atm pressure or positive pressure. In some embodiments, solubility is determined under approximately 1 atm pressure.

In some embodiments, solubility can be determined using one of the two approaches: thermodynamic or kinetic solubility. Thermodynamic solubility refers to traditional method wherein the compound is weighed in a particular solvent (buffer) and dissolved analyte is measured after reach equilibrium. Kinetic solubility is determined by preparing a concentrated stock solution comprising the solute in an organic solvent (e.g., DMSO), after which the stock solution is diluted in an aqueous buffer (e.g., PBS) to a desired concentration. In some embodiments, solubility can be determined by HPLC-UV or LC-MS/MS after filtration or spin-down to remove the insoluble.

In some embodiments, thermodynamic solubility is determined using a Fasted State Simulated Intestinal Fluid (FaSSIF) media or simulated gastric fluid (SGF) media. In some embodiments, thermodynamic solubility is determined using a FaSSIF media. In some embodiments, the thermodynamic solubility of the compound is greater than 0.8 mg/ml, greater than 0.9 mg/ml, greater than 1 mg/ml, greater than 1.1 mg/ml, greater than 1.5 mg/ml or greater than 2.0 mg/ml at pH 6.4 to 6.5, at 37° C., in a FaSSIF media. In some embodiments, the thermodynamic solubility of the compound is greater than 0.8 mg/ml, greater than 0.9 mg/ml, greater than 1 mg/ml, greater than 1.1 mg/ml, greater than 1.5 mg/ml or greater than 2.0 mg/ml at pH 6.4 to 6.5, at 37° C., in a FaSSIF media. In some embodiments, the thermodynamic solubility of the compound is greater than 1.5 mg/ml at pH 6.4 to 6.5, at 37° C., in a FaSSIF media. In some embodiments, the thermodynamic solubility of the compound is greater than 2 mg/ml at pH 6.4 to 6.5, at 37° C., in a FaSSIF media. In some embodiments, the thermodynamic solubility was determined after 12 hours, after 18 hours or after 24 hours. In some embodiments, the thermodynamic solubility of the compound is greater than 1 mg/ml at pH 6.4 to 6.5, at 37° C., in a FaSSIF media after 24 hours.

In some embodiments, the disclosure provides a compound of Formula I wherein Y* is a pharmaceutically acceptable cation, wherein the solubility of the compound of Formula I is greater than 1 mg/ml at pH 6.4 to 6.5, at 37° C., in a FaSSIF media. In some embodiments, the disclosure provides a compound of Formula I wherein Y* is a single atom cation with a +1 charge, and wherein the solubility of the compound of Formula I is greater than 1 mg/ml at pH 6.4 to 6.5, at 37° C., in a FaSSIF media.

In some embodiments, the compounds of Formula I described herein have increased bioavailability. As used herein, the term “bioavailability” generally refers to the percentage (wt/wt) of the administered dose of the compound that reaches the systemic circulation. In some embodiments, bioavailability is determined for parenteral administration or oral administration. In some embodiments, bioavailability is determined for oral administration. In some embodiments, bioavailability is determined for oral administration in animals. In some embodiments, the animals are dogs, rats, and mice.

Methods of determining the percentage of the compound in systemic circulation, e.g., the percentage in the blood, are known to the skilled artisan, and include, e.g., HPLC and LC/-MS to determine the area under the curve (AUC). Methods for determining bioavailability are outlined further in Example 2 herein. In some embodiments, bioavailability can be determined in a dog. One of skill in the art will appreciate that a dog is a useful model that in some instances corresponds with human bioavailability. In some embodiments, the bioavailability of the compound in a dog is greater than 20% (wt/wt). In some embodiments, the bioavailability of the compound in a dog is between 20% to 99% (wt/wt). In some embodiments, the bioavailability of the compound in a dog is between 40% to 95% (wt/wt). In some embodiments, the bioavailability of the compound in a dog is between 60% to 95% (wt/wt). In some embodiments, the bioavailability of the compound in a dog is between 80% to 95% (wt/wt). In some embodiments, the disclosure provides a compound of wherein Y* is a pharmaceutically acceptable cation, and wherein the bioavailability of the compound of Formula I in a dog is greater than 20%. In some embodiments, the disclosure provides a compound of wherein Y* is a single atom cation with a +1 charge, and wherein the bioavailability of the compound of Formula I in a dog is greater than 20%.

In some embodiments, the bioavailability of the compound in a human is greater than 20% (wt/wt). In some embodiments, the bioavailability of the compound in a human is between 20% to 99% (wt/wt). In some embodiments, the bioavailability of the compound in a human is between 40% to 95% (wt/wt). In some embodiments, the bioavailability of the compound in a human is between 60% to 95% (wt/wt). In some embodiments, the bioavailability of the compound in a human is between 80% to 95% (wt/wt). In some embodiments, the disclosure provides a compound of wherein Y* is a pharmaceutically acceptable cation, and wherein the bioavailability of the compound of Formula I in a human is greater than 20%. In some embodiments, the disclosure provides a compound of wherein Y* is a single atom cation with a +1 charge, and wherein the bioavailability of the compound of Formula I in a human is greater than 20%.

In some embodiments, the bioavailability of the compound of Formula 1 in a dog is at least 1.5-fold greater, 2-fold greater, 2.5-fold greater, or 3.0-fold greater than the free acid form of the compound. In some embodiments, the bioavailability of the compound in a dog is at least 2-fold greater than the free acid form of the compound.

In some embodiments, the bioavailability of the compound of Formula 1 in a human is at least 1.5-fold greater, 2-fold greater, 2.5-fold greater, or 3.0-fold greater than the free acid form of the compound. In some embodiments, the bioavailability of the compound in a human is at least 2-fold greater than the free acid form of the compound.

In some embodiments, the compounds of Formula I described herein have increased crystalline stability, i.e., they remain in their crystal form for a longer time relative to the non-salt form of PP-001. In some embodiments, the compounds of Formula I described herein have increased chemical stability, i.e., they remain in their chemical form without degradation, e.g., oxidation, for a longer time relative to the non-salt form of PP-001.

In some embodiments, the compounds of Formula I can be used in a pharmaceutical composition. In some embodiments, the disclosure provides a pharmaceutical composition comprising a compound of Formula I as described herein and a pharmaceutically acceptable excipient, carrier, diluent, or combination thereof. In some embodiments, the pharmaceutical composition comprising the compounds of Formula I are suitable for oral administration, or parenteral administration, such as subcutaneous, intravenous, or intramuscular injection or infusion.

In some embodiments, the compounds of Formula I described herein are manufactured, and then combined with one or more pharmaceutically acceptable excipients shortly before administration to a subject, e.g., less than 2 weeks, less than 1 week, less than 3 days, or less than 1 day before administration to a subject.

In some embodiments, the compounds of Formula I described herein are combined with one or more pharmaceutically acceptable excipients, and then are stable for a long period of time before administration to a subject, e.g., greater than 1 day, greater than 1 week, greater than 2 weeks, greater than 3 weeks, or greater than 1 month before administration to a subject. In some embodiments, the compounds of Formula I described herein are combined with one or more pharmaceutically acceptable excipients for storage, and then solubilized in a delivery solvent for parenteral administration.

In some embodiments, the disclosure provides a method of treating an inflammatory disease or an autoimmune disease, comprising administering to a subject in need thereof an effective amount of pharmaceutically acceptable composition comprising a compound of Formula I as described herein and a pharmaceutically acceptable excipient, carrier, diluent, or combination thereof.

In some embodiments, the administration is by oral administration. In some embodiments, administration is by parenteral administration, such as subcutaneous, intravenous, or intramuscular injection or infusion. In some embodiments, administration is by infusion.

All references cited herein, including patents, patent applications, papers, textbooks and the like, and the references cited therein, to the extent that they are not already, are hereby incorporated herein by reference in their entirety.

### EXAMPLES

### Example 1A - Kinetic Solubility

All references cited herein, including patents, patent applications, papers, textbooks and the like, and the references cited therein, to the extent that they are not already, are hereby incorporated herein by reference in their entirety.

Approximately 8 mg (calculated as freebase) of each salt was weighed out into each glass vial and then 4 ml of FaSSIF was added (target concentration was 2 mg/mL). The sample was kept stirring on a magnetic stirrer at a speed of 100 RPM under 25° C. Then at the time points of initial, 0.5 hr, 1 hr, 2 hrs and 24 hrs, 0.5 mL of sample solution was transferred into 1.5 mL centrifugation tube and centrifuged at 12,000 RPM for 10 min. The supernatant was diluted by 50%ACN suitable and analyzed by HPLC.

An HPLC method of testing solubility was performed as follows in Table A:

TABLE A

HPLC Condition for PP-001 Salt Solubility Test

| | |
|---|---|
| Content | Information |
| Chromatographic Column | SunFire C18, 3.5 µm, 150 mm*4.6 mm |
| Mobile Phase | A: 0.1% TFA in water; B: 0.1% TFA in ACN |
| Gradient | MPA:MPB = 10:90 |
| Flow Rate | 0.8 mL/min |
| Column Temperature | 25° C. |
| Injection Volume | 20 µL |
| Stop Time | 8 min |
| Detection Wavelength | 255 nm, VWD |
| Dilution | 50% ACN |

The results of the Solt Solubility Test are present in Table B:

TABLE B

Solubility results of PP-001 in 4 mL FaSSIF media

| Sample | Weight (mg) | Solubility at different time points (mg/ml) | | | | | Appearance after stirring for 24 hours | Final pH |
|---|---|---|---|---|---|---|---|---|
| | | Initial | 0.5 h | 1 h | 2 h | 24 h | | |
| Na | 8.34 | 0.41 | 1.60 | 1.73 | 1.79 | 2.01 | clear solution | 6.53 |
| Mg | 8.22 | 0.33 | 1.52 | 1.51 | 1.44 | 1.01 | suspension | 6.49 |
| Ca | 8.42 | 0.34 | 0.75 | 0.74 | 0.71 | 0.70 | suspension | 6.51 |
| K | 8.69 | 0.49 | 1.70 | 2.18 | 2.19 | 2.26 | clear solution | 6.50 |
| Zn | 9.06 | 0.19 | 0.22 | 0.21 | 0.22 | 0.26 | suspension | 6.49 |
| Lys | 10.31 | 1.37 | 2.04 | 2.06 | 2.02 | 2.09 | clear solution | 6.50 |
| Meglumine | 11.00 | 1.32 | 2.01 | 1.98 | 2.00 | 1.96 | clear solution | 6.52 |
| Tromethamine | 9.89 | 1.72 | 1.99 | 1.95 | 1.96 | 2.02 | clear solution | 6.51 |

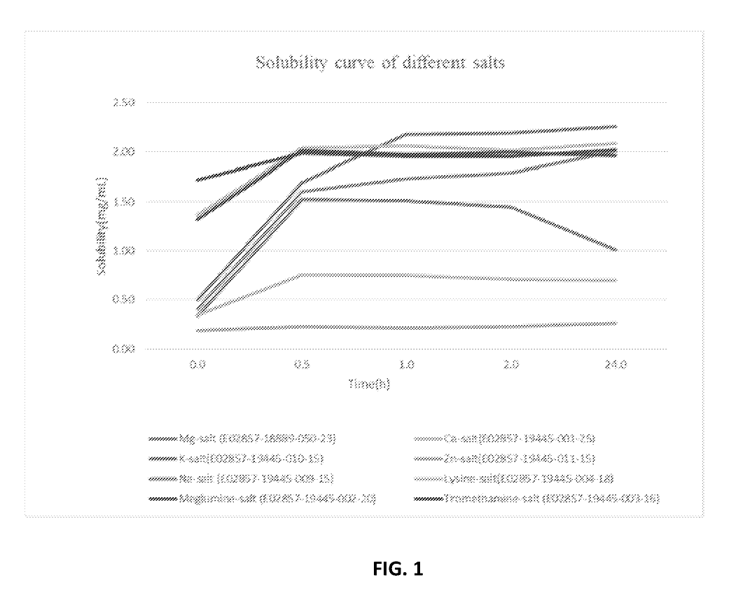

The solubility curve of the tested PP-001 salts is summarized in FIG. 1.

### Example 1B - Thermodynamic Solubility

Procedure of equilibrium solubility test: Approximately 30 mg of each salt was weighed out into each glass vial and then 3 ml of FaSSIF was added (target concentration was 10 mg/mL). As the solid was completely dissolved, add more sample to keep it saturated. The sample was kept stirring on a magnetic stirrer at a speed of 400 rpm under 37_. Then at the time point 2 hrs, 0.5 mL of sample solution was transferred into 1.5 mL centrifugation tube and centrifuged at 12,000 RPM for 10 min. The supernatant was diluted by 50%ACN suitable and analyzed by HPLC. The pH was tested after 24 hours. The results are presented in Table 1.

TABLE 1

Thermodynamic solubility of salts of PP-001 in 3 mL FaSSIF

| PP-001 Salts | Weight (mg) | Appearance after stirring for 24 hours | Solubility (mg/ml) | Final pH |
|---|---|---|---|---|
| Benzathine | 30.16 | Suspension | 0.03 | 6.43 |
| Sodium | 30.11 | Suspension | 1.85 | 6.49 |
| Potassium | 30.04 | Suspension | 2.34 | 6.49 |
| Lysine | 50.69 | Suspension | 4.65 | 6.53 |
| Meglumine | 81.23 | Suspension | 5.69 | 6.36 |
| Tromethamine | 30.13 | Suspension | 3.70 | 6.44 |

### Example 2 - Bioavailability of Salts of PP-001 in Rats

### Experimental Methods

Salts (benzathine, sodium, magnesium, calcium, potassium, zinc, lysine, meglumine, tromethamine) of PP-001 were formulated in 50 mM pH 6.8 Phosphate Buffer as 2 mg/mL suspension for single oral (PO) administration dose, volume of 2 mL/kg. Suspensions were stirred continuously prior to dose.

A total of 27 male SD rats were used (n=3). 27 male SD rats assigned to the study were divided into 9 groups and a single type of formulated PP-001 salt was orally administered to each group of rats at dose level of 4 mg/kg (2 mL/kg dose volume). The diet was provided ad libitum throughout the in-life portion of the study, except for the fasting prior to dosing through 4 hours post dose. Drinking water was available daily ad libitum to all animals.

Plasma samples were collected via the jugular vein cannulation into EDTA-K3 tubes at 0.25, 0.5, 1, 2, 4, 8, and 24 hours post-dose administration. For analytical batch of single oral administration, plasma concentrations of salts of PP-001 were determined using LC-MS/MS with a lower limit of quantitation of 1 ng/mL.

### Pharmacokinetic Analysis

The pharmacokinetic parameters of PP-001 salts were determined by non-compartmental analysis using WinNonlin Version 8.0 (Pharsight, Mountain View, CA). The area under the curve from the time of dosing to the last measurable concentration, AUC₀₋ₜ, was calculated by the linear trapezoidal rule. The area under the concentration-time curve extrapolated to infinity, AUC_{inf}, was calculated using a regression of the natural logarithm of the concentration values versus sampling time of the terminal slope (Lambda Z, k). This value is also used to calculate half-life (T_{½}) as follows:

$${\text{T}}_{1 / 2} = 0.693 / \text{k} \text{.}$$

The bioavailability was calculated as follows:

$$\text{F} \text{=} {\text{AUC\_}}_{\text{PO}} / {\text{AUC\_}}_{\text{IV}} \times {\text{Dose\_}}_{\text{IV}} / {\text{Dose\_}}_{\text{PO}} \times \text{100\%}$$

wherein PO is an oral administration and IV is an intravenous injection. The results are presented in Table 2, Table 3 and Table 4.

TABLE 2

Mean pharmacokinetic parameters of PP-001 salts after oral administration to a rat (n=3)

| PO | Group 1 (Benzathine) | | Group 2 (Sodium) | | Group 3 (Magnesium) | |
|---|---|---|---|---|---|---|
| Parameters | Mean | SD | Mean | SD | Mean | SD |
| T_{½} (hr) | 2.70 | 0.26 | 3.34 | 0.05 | 3.01 | 0.35 |
| Tₘₐₓ (hr) | 3.33 | 1.15 | 2.33 | 1.53 | 3.33 | 1.15 |
| Cₘₐₓ (ng/mL) | 1495 | 1050 | 6147 | 2838 | 3163 | 783 |
| AUCₗₐₛₜ (hr^{∗}ng/mL) | 9559 | 5751 | 32626 | 11303 | 22909 | 2471 |
| AUC_{0-∞} (hr^{∗}ng/mL) | 9593 | 5782 | 32873 | 11406 | 23029 | 2528 |
| AUC_{Extr} (%) | 0.298 | 0.156 | 0.743 | 0.088 | 0.510 | 0.304 |
| MRT_{0-∞} (hr) | 4.81 | 0.27 | 4.76 | 0.45 | 4.87 | 1.15 |
| AUCInf / D (hr^{∗}kg^{∗}ng/mL/mg) | 2398 | 1445 | 8218 | 2851 | 5757 | 632 |
| F (%) | 5.5 | 3.32 | 18.8 | 6.5 | 13.2 | 1.4 |

TABLE 3

Mean pharmacokinetic parameters of PP-001 salts after oral administration to a rat (n=3)

| PO | Group 4 (Calcium) | | Group 5 (Potassium) | | Group 6 (Zinc) | |
|---|---|---|---|---|---|---|
| Parameters | Mean | SD | Mean | SD | Mean | SD |
| T_{½} (hr) | 3.40 | 0.62 | 2.96 | 0.57 | 2.89 | 0.38 |
| Tₘₐₓ (hr) | 4.00 | 0.00 | 4.00 | 0.00 | 4.00 | 0.00 |
| Cₘₐₓ (ng/mL) | 3280 | 1017 | 1144 | 593 | 2490 | 452 |
| AUCₗₐₛₜ (hr^{∗}ng/mL) | 26827 | 9473 | 10049 | 5909 | 17033 | 4851 |
| AUC_{0-∞} (hr^{∗}ng/mL) | 27157 | 9772 | 10112 | 5948 | 17122 | 4920 |
| AUC_{Extr} (%) | 1.03 | 0.77 | 0.525 | 0.459 | 0.471 | 0.281 |
| MRT_{0-∞} (hr) | 5.55 | 0.48 | 5.31 | 0.33 | 5.33 | 0.85 |
| AUCInf / D(hr^{∗}kg^{∗}ng/mL/mg) | 6789 | 2443 | 2528 | 1487 | 4281 | 1230 |
| F (%) | 15.6 | 5.6 | 5.8 | 3.41 | 9.82 | 2.82 |

TABLE 4

Mean pharmacokinetic parameters of salts of PP-001 after oral administration to a rat (n=3)

| PO | Group 7 (Lysine) | | Group 8 (Meglumine) | | Group 9 (Tromethamine) | |
|---|---|---|---|---|---|---|
| Parameters | Mean | SD | Mean | SD | Mean | SD |
| T_{½} (hr) | 2.85 | 0.14 | 3.02 | 0.14 | 3.39 | 0.32 |
| Tₘₐₓ (hr) | 4.00 | 0.00 | 3.00 | 1.73 | 4.00 | 0.00 |
| Cₘₐₓ (ng/mL) | 2663 | 930 | 2217 | 665 | 2787 | 25 |
| AUCₗₐₛₜ (hr^{∗}ng/mL) | 18361 | 6481 | 16995 | 5884 | 21665 | 2196 |
| AUC_{0-∞} (hr^{∗}ng/mL) | 18430 | 6493 | 17083 | 5937 | 21874 | 2304 |
| AUC_{Extr} (%) | 0.388 | 0.105 | 0.488 | 0.131 | 0.930 | 0.418 |
| MRT_{0-∞} (hr) | 4.97 | 0.29 | 4.83 | 0.41 | 5.38 | 0.50 |
| AUCInf / D (hr^{∗}kg^{∗}ng/mL/mg) | 4607 | 1623 | 4271 | 1484 | 5469 | 576 |
| F (%) | 10.6 | 3.7 | 9.80 | 3.4 | 12.5 | 1.3 |

### Example 3 - Bioavailability of Salts of PP-001 in Dogs

### Experimental Methods

Based on the solubility result and bioavailability in rats result, specific salts of PP-001 were selected for determining bioavailability in dogs.

Salts (sodium, potassium, calcium, tromethamine) of PP-001 were formulated in 50 mM pH 6.8 Phosphate Buffer as 2 mg/mL suspension for single oral (PO) administration dosed volume of 2 mL/kg. Suspensions were stirred continuously prior to dose.

A total of 6 male beagle dogs were used (n=3). 6 male dogs assigned to the study were divided into 2 periods with 6 dogs per period and two dosing cycles separated by a 7-day washout period. The diet was provided ad libitum throughout the in-life portion of the study, except for the fasting prior to dosing through 4 hours post dose. Drinking water was available daily ad libitum to all animals. Formulated PP-001 salts were administered to male beagle dogs by oral administration at 4 mg/kg with dosed volume at 2 mL/kg.

Plasma samples were collected via the jugular vein cannulation into EDTA-K3 tubes at 0.25, 0.5, 1, 2, 4, 8, and 24 hours post-dose administration. For analytical batch of single oral administration, plasma concentrations of salts of PP-001 were determined using LC-MS/MS with a lower limit of quantitation of 10 ng/mL.

### Pharmacokinetic Analysis

The pharmacokinetic parameters of PP-001 salts were determined by non-compartmental analysis using WinNonlin Version 8.0 (Pharsight, Mountain View, CA). The area under the curve from the time of dosing to the last measurable concentration, AUC₀₋ₜ, was calculated by the linear trapezoidal rule. The area under the concentration-time curve extrapolated to infinity, AUC_{inf}, was calculated using a regression of the natural logarithm of the concentration values versus sampling time of the terminal slope (Lambda Z, k). This value is also used to calculate half-life (T_{½}) as follows:

$${\text{T}}_{1 / 2} = 0.693 / \text{k} \text{.}$$

The bioavailability was calculated as follows:

$$\text{F} \text{=} {\text{AUC\_}}_{\text{PO}} / {\text{AUC\_}}_{\text{IV}} \times {\text{Dose\_}}_{\text{IV}} / {\text{Dose\_}}_{\text{PO}} \times \text{100\%}$$

wherein PO is an oral administration and IV is an intravenous injection. The results are presented in Table 5.

TABLE 5

Mean pharmacokinetic parameters of salts of PP-001 after oral administration to a dog (n = 3)

| PO | Group 1 (Sodium) | | Group 2 (Potassium) | | Group 3 (Calcium) | | Group 4 (Tromethamine) | |
|---|---|---|---|---|---|---|---|---|
| Parameters | Mean | SD | Mean | SD | Mean | SD | Mean | SD |
| T_{½} (hr) | 4.24 | 0.26 | 4.15 | 0.43 | 3.76 | 0.59 | 4.13 | 0.64 |
| Tₘₐₓ (hr) | 3.00 | 1.73 | 1.17 | 0.76 | 1.67 | 0.58 | 2.67 | 1.15 |
| Cₘₐₓ (ng/mL) | 16867 | 3479 | 33967 | 9513 | 22467 | 2616 | 20467 | 2892 |
| AUCₗₐₛₜ (hr^{∗}ng/mL) | 114152 | 23961 | 177228 | 43027 | 131819 | 15890 | 104381 | 3334 |
| AUC_{0-∞} (hr^{∗}ng/mL) | 116660 | 24485 | 180263 | 44645 | 133409 | 16503 | 106528 | 2579 |
| AUC_{Extr} (%) | 2.16 | 0.34 | 1.59 | 0.73 | 1.16 | 0.65 | 2.03 | 0.98 |
| MRT_{0-∞} (hr) | 5.28 | 0.10 | 4.34 | 0.52 | 4.38 | 0.41 | 5.38 | 0.43 |
| AUCInf / D (hr^{∗}kg^{∗}ng/mL/mg) | 29165 | 6121 | 45066 | 11161 | 33352 | 4126 | 26632 | 645 |
| F (%) | 59.6 | 12.5 | 92.0 | 22.8 | 68.1 | 8.4 | 54.4 | 1.3 |

### Example 4. Summary

The results of Examples 1, 2 and 3 are summarized below in Table 6.

TABLE 6

| Compound | Solubility FaSSIF at 37° C., pH 6.4 to 6.5 (mg/ml) | Bioavailability (BA) in rats [%] | Increase in oral BA vs free acid in rats | Bioavailability (BA) in dogs [%] | Increase in oral BA vs free acid in dogs |
|---|---|---|---|---|---|
| PP-001 free acid | 1.2 (^{∗∗}) | 7.8 | 1 | 23 | 1 |
| Benzathine cation | 0.03 | 5.5 | 0.7 | n.d. | NA |
| Na cation | 1.85 | 18.8 | 2.4 | 59.6 | 2.6 |
| Mg cation | 1.01 (^{∗}) | 13.2 | 1.7 | n.d. | NA |
| Ca cation | 0.7 (^{∗}) | 15.6 | 2.0 | 68.1 | 3.0 |
| K cation | 2.34 | 5.8 | 0.7 | 92 | 4.0 |
| Zn cation | 0.26 (^{∗}) | 9.82 | 1.3 | n.d. | NA |
| Lysine cation | 4.65 | 10.6 | 1.4 | n.d. | NA |
| Meglumine cation | 5.69 | 9.8 | 1.3 | n.d. | NA |
| Tromethamine cation | 3.7 | 12.5 | 1.6 | 54.4 | 2.4 |

(^{∗}) solubility in supernatant of 2 mg/mL formulation at 25° C. in FASSIF after 24 hours
(^{∗∗}) solubility in supernatant of 10 mg/mL formulation at 25° C. in FASSIF after 120 min

As Table 6 demonstrates, the solubility of the various salt forms of PP-001 vary widely, and do not necessarily correlate with an increase in bioavailability. For example, PP-001 Ca cation has reduced solubility compared to PP-001 free acid, but over a 3-fold increase in bioavailability in dogs. In other instances, the PP-001 K cation has both increased solubility (>100% increase) and increased bioavailability in dogs (>400% increase).

1. A compound of Formula I 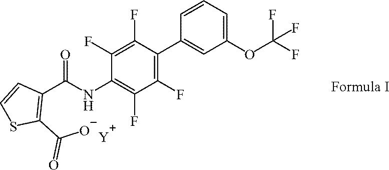
wherein Y* is
i) a single atom cation with a +1 charge;
ii) a single atom cation with a +2 charge;
iii) a carboxyalkylammonium cation, optionally substituted with one or more hydroxyl or amino groups;
iv) a dialkylammonium cation, optionally substituted with one or more hydroxyl groups; or
v) an alkylammonium cation, optionally substituted with one or more hydroxyl groups.

2. The compound of claim 1, wherein Y* is a single atom cation with a +1 charge, and the single atom cation with a +1 charge is a sodium cation or a potassium cation.

3. (canceled)

4. The compound of claim 1, wherein Y* is a single atom cation with a +2 charge, and the single atom cation with a +2 charge is a calcium cation, a magnesium cation, or a zinc cation.

5. (canceled)

6. (canceled)

7. The compound of claim 1, wherein Y* is a carboxyalkylammonium cation, and the carboxyalkylammonium cation is a carboxy(C₃-C₆) alkylammonium cation.

8. The compound of claim 7, wherein the carboxy(C₃-C₆)alkylammonium cation is a carboxypentylammonium cation.

9. (canceled)

10. The compound of claim 8, wherein the carboxypentylammonium cation is lysine.

11. (canceled)

12. The compound of claim 1, wherein Y* is a dialkylammonium cation, and the dialkylammonium cation is a (C₃-C₆)dialkylammonium cation.

13. The compound of claim 12, wherein the (C₃-C₆)dialkylammonium cation is a hexylammoniumalkyl cation.

14. The compound of claim 13, wherein the hexylammoniumalkyl cation is a hexylammoniummethyl cation.

15. (canceled)

16. The compound of claim 14, wherein the hexylammoniummethyl cation is meglumine.

17. (canceled)

18. The compound of claim 1, wherein Y* is an alkylammonium cation, and the alkylammonium cation is a (C₃-C₆)alkylammonium cation.

19. The compound of claim 18, wherein the (C₃-C₆)alkylammonium cation is a butylammonium cation.

20. The compound of claim 19, wherein the butylammonium cation is a tert-butylammonium cation.

21. (canceled)

22. The compound of claim 20, wherein the tert-butylammonium cation is tromethamine.

23. The compound of claim 1, wherein the solubility of the compound of Formula I is greater than 1 mg/ml at 37° C., pH 6.4 to 6.5 and 1 atm pressure.

24. The compound of claim 1, wherein the bioavailability of the compound of Formula I in a dog is greater than 20%.

25-27. (canceled)

28. A pharmaceutical composition comprising:
a. a compound of claim 1; and
b. a pharmaceutically acceptable excipient, carrier, diluent, or combination thereof.

29. A method of treating an inflammatory disease or an autoimmune disease comprising administering to a subject in need thereof an effective amount of pharmaceutically acceptable composition comprising:
a) a compound of Formula I 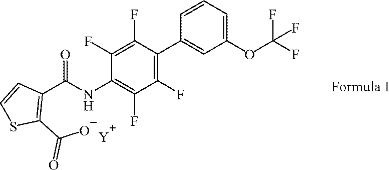
wherein Y* is
i) a single atom cation with a +1 charge;
ii) a single atom cation with a +2 charge;
iii) a carboxyalkylammonium cation, optionally substituted with one or more hydroxyl or amino groups;
iv) a dialkylammonium cation, optionally substituted with one or more hydroxyl groups; or
v) an alkylammonium cation, optionally substituted with one or more hydroxyl groups; and
b) a pharmaceutically acceptable excipient, carrier, diluent, or combination thereof.

30. The method of claim 29, wherein the administering is by oral administration.

31. (canceled)

32. (canceled)

33. A compound of Formula I 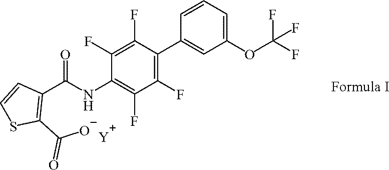
wherein Y* is a pharmaceutically acceptable cation, wherein the solubility of the compound of Formula I is greater than 1 mg/ml at pH 6.4 to 6.5, at 37° C., in a FaSSIF media.
